(19) United States
(12) Patent Application Publication
(10) Pub. No.: US 2019/0110937 A1
(43) Pub. Date: Apr. 18, 2019

(54) INCONTINENCE PAD CONFIGURED FOR USE WITH A WATER-SOLUBLE ADHESIVE

(71) Applicant: amysquared, LLC, Manitou Springs, CO (US)

(72) Inventors: Cathy Lynn Amy, Shreveport, LA (US); Amy Louise Edwards, Manitou Springs, CO (US)

(21) Appl. No.: 16/142,836

(22) Filed: Sep. 26, 2018

Related U.S. Application Data
(60) Provisional application No. 62/563,282, filed on Sep. 26, 2017.

(51) Int. Cl. A61F 13/15; A61F 13/56; A61F 13/514; A61F 13/505; A61F 13/84; A61L 15/58; A61L 15/24

(52) U.S. Cl. CPC A61F 13/15268; A61F 13/5611; A61F 13/51405; A61F 13/505; A61F 13/8405; A61F 2013/1556; A61L 15/585; A61L 15/24; A61F 13/15211; A61F 2013/15276; A61F 2013/15235; A61L 15/58

(57) ABSTRACT

Various embodiments for a washable and reusable absorbent incontinence pad are disclosed. An incontinence pad may include a top layer configured to be positioned against skin of a wearer, a bottom layer configured to be positioned against an undergarment, a moisture-absorbing inner layer, and a water-resistant inner layer. The moisture-absorbing inner layer and the water-resistant inner layer may be positioned between the top layer and the bottom layer. A raised coating layer may also be laminated on the bottom layer, where the raised coating layer is configured to retain a water-soluble adhesive thereon to secure the incontinence pad to an undergarment. The water-soluble adhesive may be configured to fully wash away from the pad and garment and make the pad removable from the undergarment without damaging the undergarment or leaving a substantial amount of residue thereon.

### CROSS-REFERENCE TO RELATED APPLICATION

This application claims the benefit of and priority to U.S. Provisional Patent Application No. 62/563,282 entitled “INCONTINENCE PAD CONFIGURED FOR USE WITH A WATER-SOLUBLE ADHESIVE, Sep. 26, 2017, the contents of which being incorporated by reference in their entirety herein.

### BACKGROUND

Incontinence pads are frequently worn by females to treat incontinence and are commonly formed of moisture absorbent materials. However, pads and other similar types of undergarments are unsuitable for extended use as absorptive properties of the pad can result in the pad becoming unhygienic. For instance, many types of pads can be required to be changed frequently due to growth of bacteria or other microorganisms, causing unpleasant odor and skin irritation. Thus, single-use incontinence pads are generally more popular. Single-use incontinence pads, however, can be more expensive, may be made of materials that chafe, and can be harmful to the environment.

### SUMMARY OF THE INVENTION

Various embodiments for a washable and reusable incontinence pad are disclosed that may be used for urinary incontinence or other type of urinary or vaginal discharge. A washable and reusable incontinence pad may include, for example, a layer configured to retain a water-soluble adhesive that adheres to underwear, shapewear, or other type of garment. The water-soluble adhesive may be applied such that the adhesive washes away substantially from the incontinence pad and garment upon laundering. In some embodiments, the adhesive may be applied to a polymer-based coating situated on the back of the pad such that the pad is firmly anchored to the underwear yet easily removable without damaging underwear or leaving residue thereon.

The washable and reusable incontinence pad may comprise hydrophobic and hydrophilic layers such that urine or other discharge may be wicked or otherwise pulled and retained in an interior of the pad. Additionally, the incontinence pad may be formed in such a way that the pad does not require any specialty underwear to be worn in association with the pad. For instance, the washable and reusable incontinence pad may not require any modification to a wearer's underwear, shapewear, or other similar type of garment. Unlike other reusable pads, the incontinence pad does not use any wrap around appendages, snaps, or hooks for attaching the pad to underwear.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 is a layered perspective view of the washable and reusable incontinence pad according to various embodiments of the present disclosure.

FIG. 2 is a top view of a washable and reusable incontinence pad according to various embodiments of the present disclosure.

FIG. 3 is a bottom view of the washable and reusable incontinence pad of FIG. 1 according to various embodiments of the present disclosure.

FIG. 4 is an exploded cross-sectional views of the washable and reusable incontinence pad of FIG. 1 according to various embodiments of the present disclosure.

FIG. 5 is a cross-sectional views of the washable and reusable incontinence pad of FIG. 1 according to various embodiments of the present disclosure.

FIG. 6 is a flowchart describing an example use of the incontinence pad of FIG. 1 according to various embodiments of the present disclosure.

FIGS. 7, 8, and 9 are photographs illustrating an example application of an adhesive to the incontinence pad of FIG. 1 according to various embodiments of the present disclosure.

FIG. 10 is a photograph illustrating adhesion of the incontinence pad of FIG. 1 to a fabric according to various embodiments of the present disclosure.

FIG. 11 is a photograph illustrating an embodiment of the incontinence pad of FIG. 1 according to various embodiments of the present disclosure.

FIG. 12 is a photograph illustrating an example system comprising multiple incontinence pads according to various embodiments of the present disclosure.

### DETAILED DESCRIPTION

The present disclosure relates to an absorbent incontinence pad that is washable and reusable. As may be appreciated, the incontinence pad disclosed herein may be used to treat urinary incontinence or, in some situations, urinary or vaginal discharge. To this end, in various embodiments, the incontinence pad may include, for example, a plurality of layers, where at least one of the plurality of layers is configured to receive and retain an application of a water-soluble adhesive suitable to adhere the incontinence pad to underwear, shapewear, or other type of undergarment. The properties of the layer configured to retain the water-soluble adhesive, and the properties of the water-soluble adhesive itself, may cause the water-soluble adhesive to wash away fully from the incontinence pad when washed or laundered. In some embodiments, the adhesive may be applied to a polymer-based coating situated on a bottom of the incontinence pad such that the incontinence pad is firmly anchored to the underwear yet easily removable without damaging underwear or leaving residue thereon.

In the following discussion, a general description of a washable and reusable incontinence pad and its components is provided, followed by a discussion of the operation of the same.

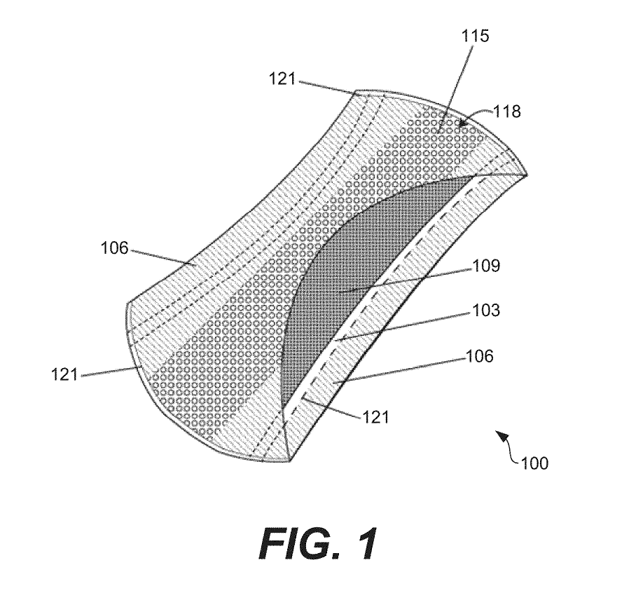

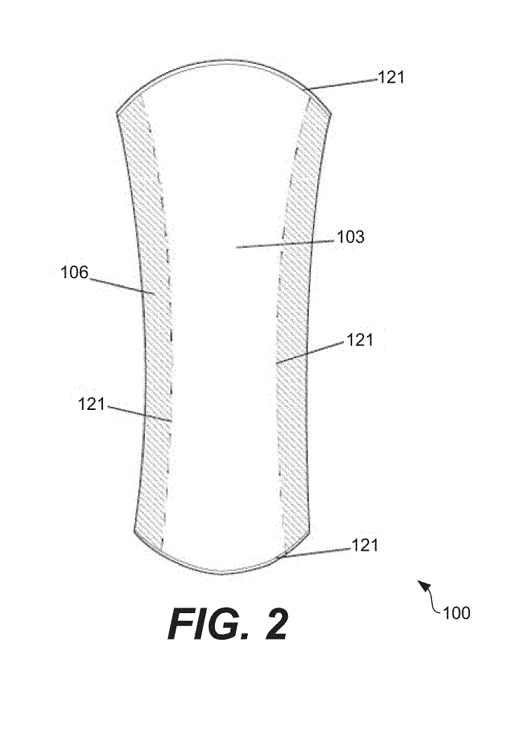

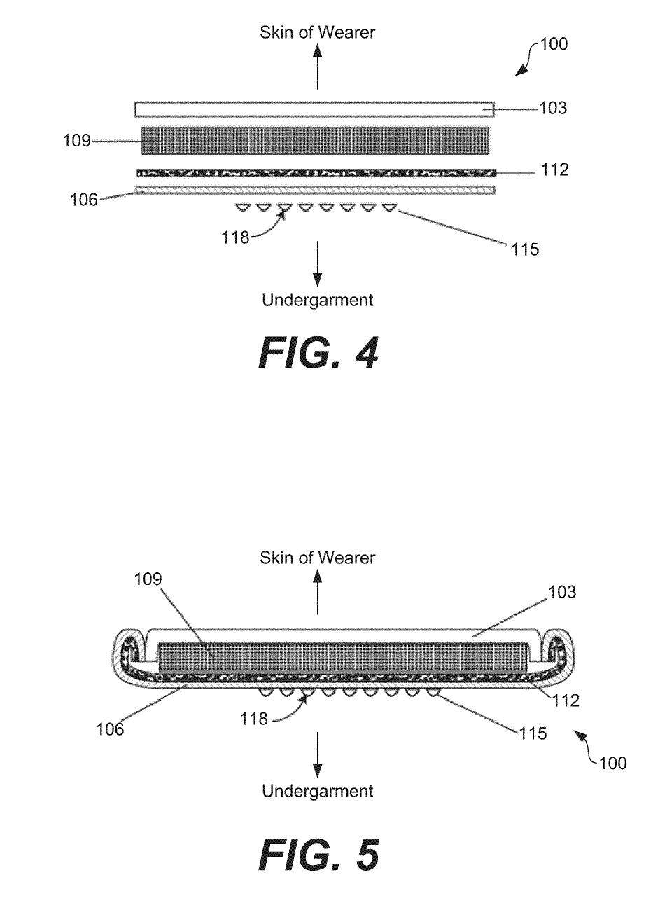

Referring now to FIGS. 1-5, an incontinence pad 100 is shown according to various embodiments. As shown in the layered perspective view of FIG. 1 and in the cross-sectional views of FIGS. 4 and 5, the incontinence pad 100 may be described as including five layers for explanatory purposes, although other numbers of layers may be employed. The layers of the incontinence pad 100 may include, for example, a top layer 103 configured to contact skin of a wearer and a bottom layer 106 configured to contact an undergarment of the wearer. Generally, the incontinence pad 100 may be formed such that urine or other discharge is wicked from the skin of the wearer and retained in an interior of the incontinence pad 100, thereby retaining odor and preventing rashes due to excessive moisture on portions of the incontinence pad 100 that contact the skin of the wearer (e.g., the top layer 103). To this end, the top layer 103 may comprise water absorbent, hydrophilic, and/or anti-microbial fabric such that the top layer 103 wicks and absorbs moisture into the interior layers of the incontinence pad 100, as will be described.

As the top layer 103 is configured to come into contact with the skin of the wearer, the top layer 103 may be made of fibers that are soft to the touch and therefore less likely to chafe to help prevent skin irritation. Accordingly, in various embodiments, the top layer 103 may include a fabric comprising at least one of: bamboo fibers and organic cotton fibers. Additionally, the top layer 103 may be formed to act as a hypo-allergenic and antimicrobial absorbent layer. The bottom layer 106, on the other hand, may include and a leak-resistant backing.

Disposed between the top layer 103 and the bottom layer 106, the incontinence pad 100 may include a moisture-absorbing inner layer 109 and a water-resistant inner layer 112. The moisture-absorbing inner layer 109 may be positioned below and adjacent to the top layer 103 to wick moisture from the top layer 103 into the interior of the incontinence pad 100. Similar to the top layer 103, the moisture-absorbing inner layer 109 may be formed of absorbent, hydrophilic, and anti-microbial fabric. The water-resistant inner layer 112, on the other hand, may be positioned above and adjacent to the bottom layer 106, thereby causing moisture to be retained in the moisture-absorbing inner layer 109. As may be appreciated, by retaining moisture in the moisture-absorbing inner layer 109, moisture is prevented from traveling to the bottom layer 106 or the undergarment. The water-resistant inner layer 112 may be formed of hydrophobic textiles and have a lower wickability than fibers or fabrics of the other layers. The moisture-absorbing inner layer 109 may be formed of hydrophilic textiles and have a higher wickability than fibers or fabrics of the other layers. In some embodiments, the water-resistant inner layer 112 may be laminated to an interior of the bottom layer 106.

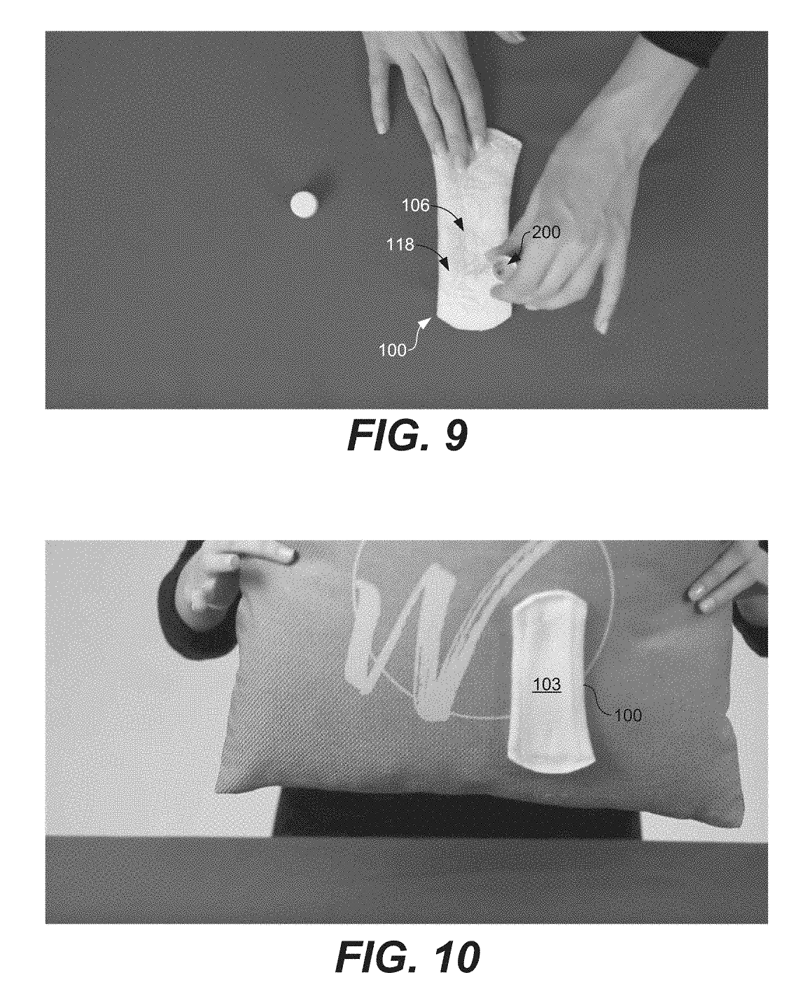

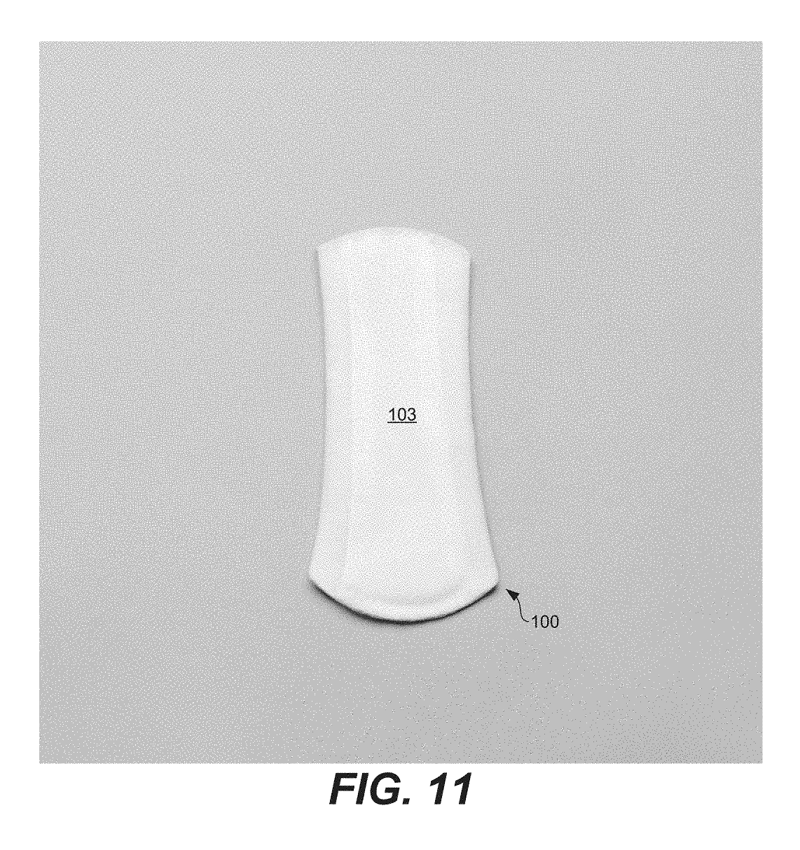

Additionally, in some embodiments, a raised coating layer 115 may be positioned on a surface of the bottom layer 106 to come into contact with an undergarment of the wearer. In some embodiments, the raised coating layer 115 includes a plurality of projections 118 projecting downward from a surface of the bottom layer 106. Also, in various embodiments, the raised coating layer 115 may be in other arrangements that project downward from the bottom layer 106. For instance, as shown in FIG. 9, the raised coating layer 115 includes a floral arrangement raised from a surface of the bottom layer 106, although other arrangements may be employed.

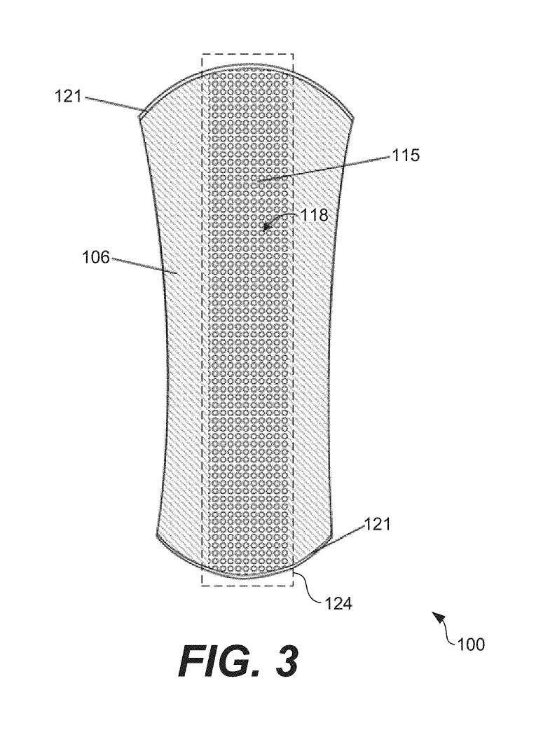

Additionally, in some embodiments, the plurality of projections 118 of the raised coating layer 115 may be polymer-based projections configured to receive and retain a coating of adhesive thereon. As shown in FIG. 4 and FIG. 5, the projections 118 may include semi-circular projections. Referring specifically to FIG. 3, the projections 118 may be arranged in a central region 124 of the incontinence pad 100 in some embodiments. Notably, the projections 118 form a rectangular and symmetrical pattern extending along a length of the central region 124, which may improve the adherence of the incontinence pad 100 to an undergarment. In the embodiment shown in FIG. 9, however, the raised coating layer 115 is shown in a floral arrangement as a decorative design. However, the raised coating layer 115 aids in the retention of an adhesive, as will be described.

The moisture-absorbing inner layer 109 may be enveloped by a fabric casing formed of the top layer 103 and the bottom layer 106. For instance, the textile materials of the top layer 103 and the bottom layer 106 may be sewn together using stitching 121 along all sides to enclose the moisture-absorbing inner layer 109. In some embodiments, the water-resistant inner layer 112 may be bonded or otherwise attached to the bottom layer 106 before or in place of sewing. Various stitching techniques may be used, such as, for example, ultrasonic welding. Additionally, stitching 121 may be applied to an interior of the incontinence pad 100 to hold the layers in place.

The projections 118 of the raised coating layer 115 may include slightly-raised polymer-based projections to form various designs and shapes that may facilitate friction and adhesion to secure the incontinence pad 100 to an undergarment of a wearer. For instance, the incontinence pad 100 shown in FIG. 9 illustrates a floral design. Additionally, the projections 118 may be applied to the bottom layer 106 of the pad to provide a suitable surface for application of an adhesive. The arrangement of the projections 118 may assume different patterns, for example, to conform to different types of undergarments and/or to provide different aesthetics to the incontinence pad 100.

The materials of the top layer 103, the bottom layer 106, the moisture-absorbing inner layer 109, and the water-resistant inner layer 112 may be constructed or otherwise formed from various materials, such as natural and synthetic textiles and other materials. For instance, the bottom layer 106 may comprise natural or synthetic textile fibers or other material. In one embodiment, the bottom layer 106 may comprise bamboo, cotton, organic cotton, polyester, a combination thereof, or other textile fibers.

In some embodiments, the water-resistant inner layer 112 may be formed or molded from polyurethane that is laminated to the bottom layer 106. Similar to the bottom layer 106, the top layer 103 may comprise natural or synthetic textile fibers or other material. In one embodiment, the top layer 103 may include bamboo, cotton, organic cotton, polyester, or other textile fibers.

The moisture-absorbing inner layer 109 may include an anti-microbial blend of cellulosic fibers which may include, for example, bamboo, cotton, organic cotton, polyester or other suitable natural or synthetic textile material. The projections 118 or other portion of the raised coating layer 115 may be formed of or have an outer layer of a polymer-based material. The polymer-based material may include, for example, polysiloxane, silicone, other suitable natural or synthetic material, or a combination thereof.

The washable and reusable incontinence pad 100 described herein may be formed in such a way that the incontinence pad 100 does not require any specialty underwear to be worn in association with the pad. Additionally, the incontinence pad 100 may not require any modification to a wearer's underwear, shapewear, or other similar type of garment. Unlike other reusable pads, the incontinence pad 100 may not comprise any wrap-around appendages, snaps, hooks, or other similar means for attaching the incontinence pad 100 to an undergarment.

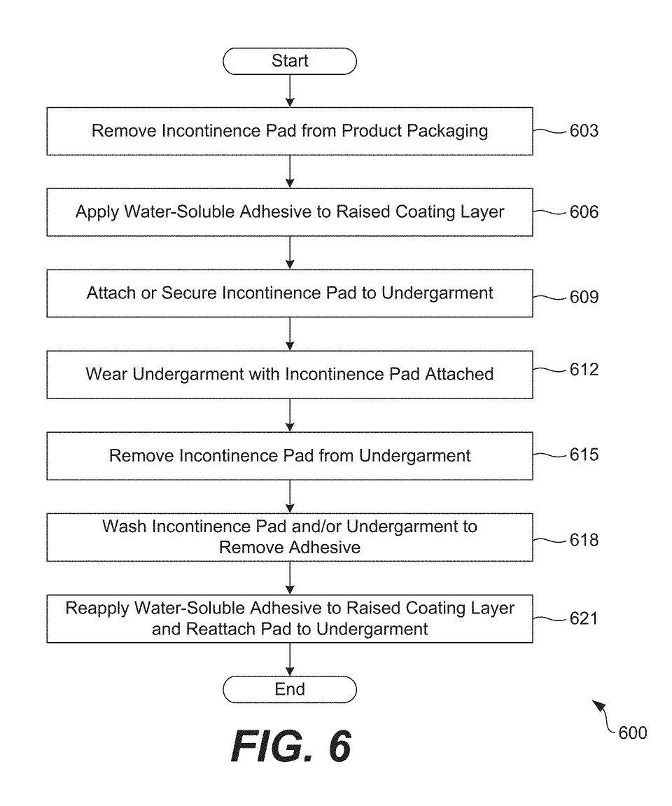

Referring next to FIG. 6, shown is a flowchart 600 that provides an example of operation or use of the incontinence pad 100 according to various embodiments. It is understood that the flowchart of FIG. 6 provides merely an example of the many different types of arrangements that may be employed to wear the incontinence pad 100 as described herein.

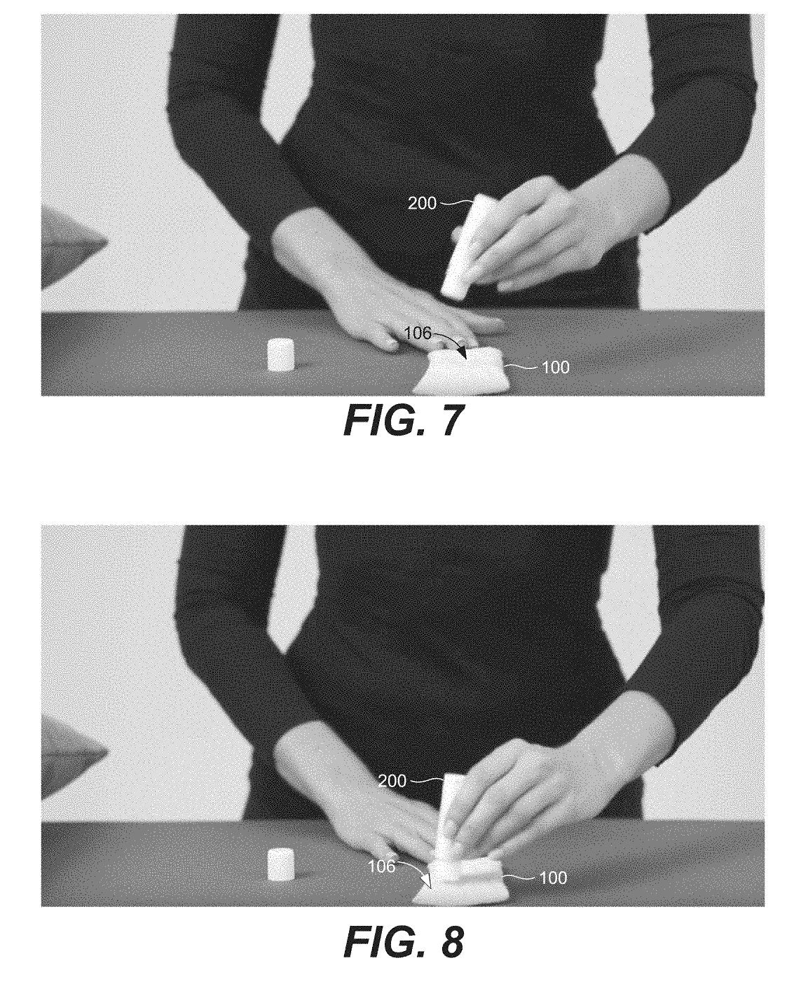

Beginning with step 603, a person may remove the incontinence pad 100 from the product packaging. In some embodiments, the incontinence pad 100 may not have an adhesive (e.g., a water-soluble adhesive) already applied to the raised coating layer 115. Thus, in step 606, the person may apply to the water-soluble adhesive to the raised coating layer 115. FIGS. 7, 8, and 9 illustrate the application of an adhesive to the raised coating layer 115 using an adhesive applicator 200. The adhesive applicator 200 may include the adhesive stored in an interior chamber, for instance, as a gel or wax-like body similar to lip balm.

In step 609, the incontinence pad 100 may be positioned, attached, or secured a bottom of an undergarment. FIG. 10 illustrates an application of the incontinence pad 100 to a fabric (e.g., a pillow fabric) having similar characteristics as an interior of an undergarment. While a pillow is shown for explanatory reasons, it is understood that the incontinence pad 100 will adhere similarly to an interior of an undergarment.

As may be appreciated, the adhesive may be sold along with the incontinence pad 100 or sold as a separate item. Additionally, the water-soluble adhesive may be sold in an adhesive stick (e.g., similar to a glue stick or lip balm) or other packaging that facilitates the application of the water-soluble adhesive to a layer of the incontinence pad 100. In various embodiments, the water-soluble adhesive may comprise polyvinylpyrrolidone, which is a water-soluble polymer formed from the monomer N-vinylpyrrolidone, or other adhesives with similar properties such as polyvinyl alcohol, or a blend thereof.

In step 612, the person may wear the undergarment having the incontinence pad 100 attached therein. Notable, the incontinence pad 100 does not require any modification to a wearer's underwear, shapewear, or other similar type of garment. Unlike other reusable pads, the incontinence pad 100 may not include any wrap-around appendages, snaps, hooks, or other similar means for attaching the incontinence pad 100 to an undergarment.

In step 615, after use, the person may remove the incontinence pad 100 from the undergarment. In some embodiments, the water-soluble adhesive may be applied to a polymer-based coating situated on a bottom of the incontinence pad 100 such that the incontinence pad 100 is firmly anchored to the undergarment yet easily removable without damaging the undergarment or leaving residue thereon. In the event that some residue is left in the undergarment, the residue can be easily washed off using, for example, soap and water.

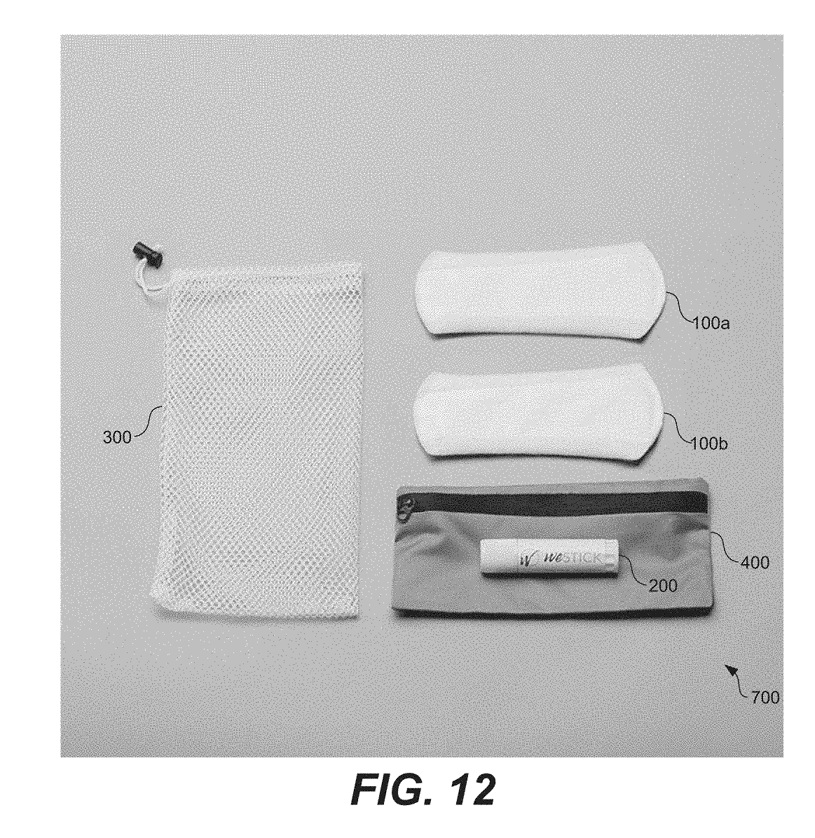

In step 618, the person may wash the incontinence pad 100 and/or the undergarment to remove the water-soluble adhesive as well as to clean the incontinence pad 100. In some embodiments, the insentience pad 100 may be placed in a porous wash bag 300 prior to placement in a washer or dryer, as shown in FIG. 12.

In step 621, the person may reapply the water-soluble adhesive, as described above with respect to step 606, and reattach the incontinence pad 100 to the undergarment for continued use. The incontinence pad 100, when clean, may be stored in a storage bag 400 or other suitable location. An example of a system 700 or a kit is shown that includes a first incontinence pad 100a, a second incontinence pad 100b, an adhesive applicator 200, a porous wash bag 300 (having a draw string, for example), and a storage bag 400 (which may be more discrete than the porous wash bag 100 and include a zipper, for example).

In addition to providing a highly absorbent pad that is washable and reusable, the incontinence pad 100 may be configured to wick and retain moisture in an interior of the incontinence pad 100, which may retain fluid and odor while minimizing leakage to the undergarment. Additionally, the incontinence pad 100 wicks urine or other bodily fluid away from the skin of the wearer to minimize rashes or sores. In some embodiments, the absorbent layers of the incontinence pad 100 may be enclosed in a hydrophobic layer, such as a polypropylene or polyethylene film, to prevent urine or other bodily fluid from coming into contact with the skin or clothing while minimizing leakage and retaining odor.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, the following is claimed:

1. An incontinence pad that is washable and reusable, comprising:
a top comfort layer configured to be positioned against skin of a wearer;
a bottom layer;
a moisture-absorbing inner layer configured to wick moisture from the top comfort layer and retain the moisture in the moisture-absorbing inner layer;
a water-resistant inner layer, wherein the moisture absorbing inner layer and the water-resistant inner layer are positioned between the top layer and the bottom layer; and
a raised coating layer positioned on the bottom layer configured to contact an undergarment of the wearer, the raised coating layer being configured to retain a water-soluble adhesive thereon and adhere the incontinence pad to an undergarment.

2. The incontinence pad of claim 1, wherein the water-soluble adhesive is configured to fully wash away from the disposable incontinence pad and the undergarment when washed or laundered.

3. The incontinence pad of claim 1, wherein the raised coating layer comprises a plurality of polymer projections.

4. The incontinence pad of claim 3, wherein the water-soluble adhesive is applied to an outer surface of the plurality of polymer projections and retained thereon.

5. The incontinence pad of claim 3, wherein the plurality of polymer projections are semi-circular projections, the plurality of polymer projections being in a rectangular and symmetrical arrangement in a central region of the bottom layer of the incontinence pad.

6. The incontinence pad of claim 4, wherein the water-soluble adhesive is configured such that the incontinence pad is removable from the undergarment without damaging the undergarment or leaving a substantial amount of residue on the undergarment.

7. The incontinence pad of claim 1, wherein the raised coating layer is laminated on the bottom layer.

8. The incontinence pad of claim 1, wherein the moisture-absorbing inner layer is positioned adjacent to the top layer and the water-resistant inner layer is positioned adjacent to the bottom layer.

9. The incontinence pad of claim 1, wherein at least a portion of the incontinence pad is formed using bamboo fibers, cotton fibers, organic cotton fibers, polyester fibers, or a combination thereof.

10. The incontinence pad of claim 1, wherein the moisture-absorbing inner layer comprises anti-microbial fibers.

11. The incontinence pad of claim 1, wherein the water-soluble adhesive comprises polyvinylpyrrolidone (PVP), polyvinyl alcohol (PVA), blended PVA/PVP, or other similar adhesives.

12. A method, comprising:
providing an incontinence pad that is washable and reusable, wherein the incontinence pad comprises:
a top layer configured to be positioned against skin of a wearer;
a bottom layer configured to be positioned against an undergarment;
a moisture-absorbing inner layer; and
a water-resistant inner layer, wherein the moisture absorbing inner layer and the water-resistant inner layer are positioned between the top layer and the bottom layer; and
a raised coating layer positioned on the bottom layer, the raised coating layer configured to retain a water-soluble adhesive thereon to secure the incontinence pad to an undergarment.

13. The method of claim 12, further comprising applying the water-soluble adhesive to a plurality of projections on the raised coating layer.

14. The method of claim 13, further comprising securing the raised coating layer to the undergarment after the water-soluble adhesive is applied to the plurality of projections.

15. The method of claim 14, further comprising:
washing the incontinence pad;
re-applying the water-soluble adhesive to the plurality of projections of the raised coating layer; and
re-securing the raised coating layer to the undergarment after the water-soluble adhesive is applied to the plurality of projections.
